(19) United States
(12) Patent Application Publication
(10) Pub. No.: US 2026/0251666 A1
(43) Pub. Date: Aug. 27, 2026

(54) REAGENT PEN TESTING DEVICE AND METHOD

(71) Applicant: Probitas LLC, Lincoln, NE (US)

(72) Inventor: Timothy Becker, Fairview, NC (US)

(73) Assignee: Probitas LLC, Lincoln, NE (US)

(21) Appl. No.: 19/060,424

(22) Filed: Feb. 21, 2025

(51) Int. Cl. G01N 33/94; G01N 21/78; G01N 31/22

(52) U.S. Cl. CPC G01N 33/946; G01N 21/78; G01N 31/22; G01N 33/9413; G01N 33/942; G01N 33/9486

(57) ABSTRACT

A device (10) to test for the presence of a predetermined substance. The device has a cap (30), a flexible reagent holder (40) reversibly attached to the cap, a breakable ampule (41) contained within the reagent holder, and a test tube (20) reversibly attached to the cap. The breakable ampule contains a reagent liquid (42). The test tube removably contains the flexible reagent holder and has at least one transparent area wherein a color change can be seen. The breakable ampule releases the reagent liquid when the flexible reagent holder is manipulated so that the reagent fluid can be mixed with a powder (60) in the test tube. If the powder contains the predetermined substance then a predetermined color is manifested. The cap may have knobs (36) to crush a pill or tablet, and/or a serrated edge (33) to scrape a pill or tablet, to provide the powder for testing.

### BACKGROUND

The diagnostic identification of medication and pharmaceutical products has become an increasingly important public health and safety concern for the U.S. population. Whether for scientific identification or diagnostic analysis, the ability to discern the presence of active ingredient(s) in a medication is imperative, particularly in the context of the large-scale pharmaceutical shortages and questioned efficacy of medication distributed through national or regional pharmaceutical outlets.

The ability to discern the presence of active ingredient(s) in a medication is also imperative during disasters when power is out for prolonged periods, and when the supply chain is disrupted for weeks or even months.

The ability to discern the presence of active ingredient(s) in a medication is also useful when the expiration date on a medication has passed but a person needs the medication sooner, rather than later, but there is no ability to quickly go and get a fresh medication, or have one quickly brought to the person, such as when the person cannot leave because of icy or snowy roads, cannot drive at night, the pharmacies have closed, etc.

The ability to discern the presence of illicit drug(s), and the identity of the illicit drug(s), is also useful for law enforcement, especially in protecting law enforcement officers and first responders. For example, when, as a result of an accident, a traffic stop, or an emergency call, law enforcement officers and first responders find an unknown substance. Is it “just” an illegal substance, like cocaine or heroin, or is it a deadly illegal substance, such as fentanyl. The ability to quickly determine the nature of the substance and take proper precautions when handling it can be the difference between life, injury, or even death.

The landscape of testing and identifying pharmaceuticals and medication has been generally limited to professional laboratory testing—using testing methods such as Gas Chromatography and Mass Spectrometer (GC-MS), Liquid Chromatography and Mass Spectrometer (LC-MS), Nuclear Magnetic Resonance (NMR) spectroscopy, Fourier Transform Infrared Spectroscopy (FTIR), thin layer chromatography (TLC) testing, and at-home options, such as reagent testing. From the perspective of the consumer, the laboratory options can be expensive and time consuming (taking weeks or more to receive results). The at-home options can have complicated testing processes, require multiple testing items and components for conducting tests, and have the constraint of limited shelf lives of the chemicals (1-1.5 years maximum if kept in optimal conditions, i.e., refrigerated or frozen).

### SUMMARY

A device to test for the presence of a predetermined substance. The device has a cap, a flexible reagent holder reversibly attached to the cap, a breakable ampule contained within the reagent holder, and a test tube reversibly attached to the cap. The breakable ampule contains a reagent liquid. The test tube removably contains the flexible reagent holder and has at least one transparent area wherein a color change can be seen. The breakable ampule releases the reagent liquid when the flexible reagent holder is manipulated so that the reagent fluid can be mixed with a powder in the test tube. If the powder contains the predetermined substance then a predetermined color is manifested. The cap may have knob to crush a pill or tablet, and/or a serrated edge to scrape a pill or tablet, to provide the powder for testing.

### BRIEF DESCRIPTION OF THE DRAWING

FIG. 1 is a side perspective view of an embodiment of the device.

FIG. 2 is a front perspective view of the device.

FIG. 3 is a side perspective view of the reagent holder.

FIG. 4 is a rear perspective view of the device.

FIG. 5 is a side view of the device without the cap.

FIG. 6 is a side perspective view of the device with the cap.

FIG. 7 is a perspective view showing an ampule within the reagent holder.

FIG. 8 is a perspective view of the ampule.

FIG. 9 is an exploded view of the test tube, the cap, and the reagent holder.

FIG. 10 is an illustration showing one or more drops of the reagent liquid being applied to a substance in the test tube.

### DETAILED DESCRIPTION

In this Detailed Description references are made to the accompanying Drawing, which form a part hereof, and that show by way of illustration various configurations, embodiments, implementations, or examples.

A diagnostic device is disclosed that solves these drawbacks, by providing an inexpensive, simple identification test, with an extended 3-year shelf life, without the need for refrigeration of any kind. This provides a low-cost option, prepackaged in an easy-to-use, portable, all-in-one container, and solves the issues of timeliness, costliness, portability, usability, and lifespan is disclosed herein.

The diagnostic device is a user-friendly, compact, at-home substance checking test kit packaged for convenience. The test kit provides individuals with comprehensive knowledge about the medication they plan to consume. The test kit incorporates reagent chemicals designed to change color upon contact with specific pharmaceutical substances, ensuring the presence of the desired active ingredient. In an embodiment, the test kit is in the form of a pioneering pen or bottle for a single-use reagent test.

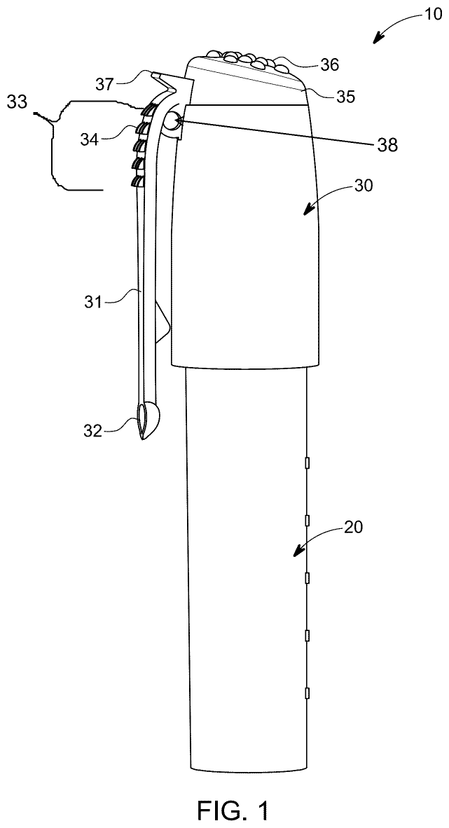

FIG. 1 is a side perspective view of an embodiment of the device 10 and depicts the device 10 in a compact (closed) position. In the compact position, the test tube 20 is reversibly fastened to the cap 30. In this embodiment, the device 10 has a similar appearance to a pen, bottle, or small flashlight.

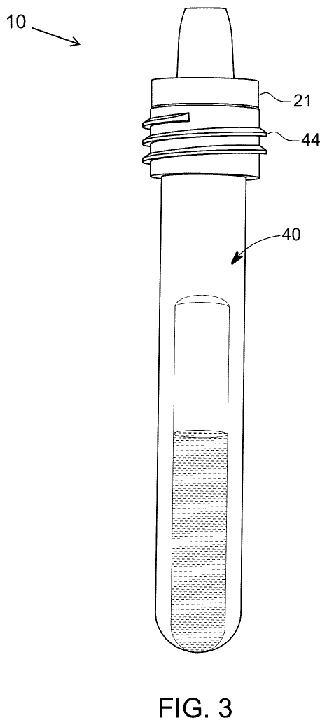

In some embodiments, the test tube 20 and cap 30 are made of hard polypropylene (PP) material, and the reagent holder 40 (FIG. 3) is made of a low-density polyethylene (LDPE) material or made of a linear low-density polyethylene (LLDPE) material.

The cap 30 has a clip 31 with a measuring end 32 designed to measure a set amount of a substance to be tested. In some embodiments, the measuring end 32 has a 10-milligram measuring spoon for a powder, powder mixture, or crushed pill.

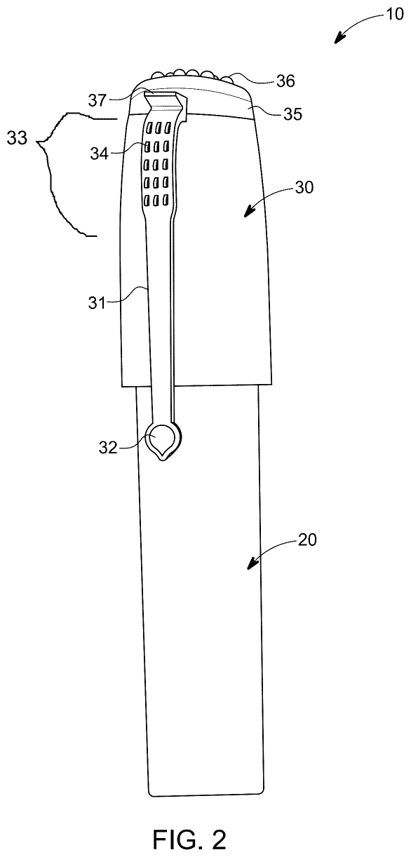

In some embodiments, the clip 31 has an end that has a serrated edge 33. One application of the serrated edge 33 is to scrape pills to produce a powder from the pill. In some embodiments, the serrated edge 33 is located along the clip 31 and has a slightly larger width than the rest of the clip 31 (best seen in FIG. 2), to accommodate multiple rows of serrated teeth 34. In an embodiment, the serrated edge 33 is approximately a third of the full length of the clip 31. In other embodiment, the serrated edge 33 is on the cap 30 in a spot other than on the clip 31.

In an embodiment, the cap 30 has a knobby end 35 that is designed for crushing up chunks of pills or other medications. In some embodiments, this knobby end 35 has knobs 36 covering the full surface of the top of the cap 30, with small spacing in between. In other embodiments, the knobby end 35 has only a small section of knobs for crushing and is not covering the full surface of the cap 30. In other embodiments, the knobby end 35 has a texture other than round knobs 36, but it is still used as a crushing mechanism.

The clip 31 can be longer or shorter than the cap 30. The cap 30 is removable from the test tube 20, allowing the test tube 20 and the reagent holder 40 to be accessible, and/or allowing a user of the device to better utilize the scooping feature of the measuring end 32, the serrated edge 33, and the knobby end 35. In an embodiment, top part of the cap 30 can detach, with the clip 31, from the rest of the cap 30 to provide for convenient use of the clip 31 and serrated edge 33.

In some embodiments, there is a tab 37 above the clip 31 that fastens the clip 31 to the cap 30. A user may use the tab 37 to remove the clip 31 from the cap 30.

In some embodiments, a portion of the cap 30 may also be removed that contains the knobby end 35. The length of the removable top section of the cap 30 that contains the knobby end 35 may be longer if desired.

In an embodiment, only the clip 31 is removable.

In an embodiment, the tab 37 is located on the cap 30 away from the knobby end 35.

In some embodiments, the tab 37 can be used as a serrated edge.

In an embodiment, the cap 30 also has an eyelet 38, which can be used with a cord (not shown) to provide for convenient carrying of the device 10. For example, the cord may be long enough to go around the neck of the user or short enough to go around the wrist of the user.

FIG. 2 is a front perspective view of the device 10. In an embodiment, the serrated edge 33 has five rows of serrated teeth 34 in three columns, equaling a total of fifteen serrated teeth 34. There can be more than, or fewer than, five rows. Also, there can be more than, or fewer than, three columns. Further, the size of the serrated teeth 34 can be adjusted to achieve a desired granularity of the substance to be tested.

When the cap 30 is attached to the test tube 20 and the reagent holder 40 the device 10 considered to be in the compact or transportable position. When the cap 30 is detached from the test tube 20 and the reagent holder 40 the device 10 is considered to be in the open or use position.

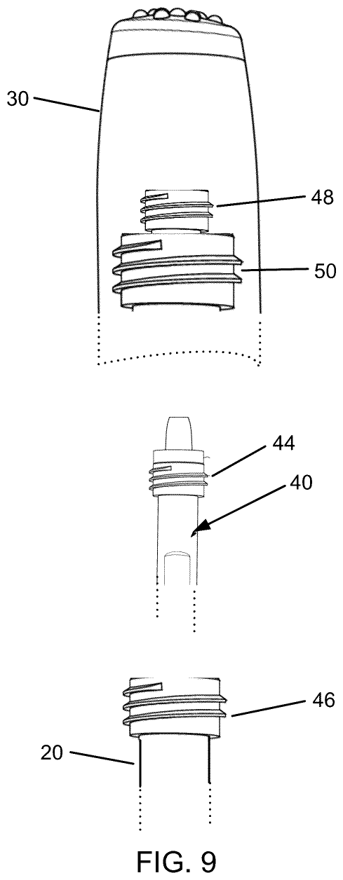

FIG. 3 is a side perspective view of the reagent holder 40. In the compact position, the reagent holder 40 is held within the test tube 20, which are fastened to the cap 30 as discussed below with respect to FIG. 9. In some embodiments, the test tube 20 is clear allowing both the test tube 20 and the reagent holder 40 to be visible, even while the pen is closed and the cap 30 fastened to the test tube 20.

In an embodiment, the cap 30 is fastened to the reagent holder 40 at the neck 21 of the reagent holder 40. The cap 30 can be fastened to the reagent holder 40 by a fastener 44, that securely attaches the cap 30 to the reagent holder 40, while still allowing a user to separate the cap 30 from the reagent holder 40.

In an embodiment the fastener 44 is a threaded bushing. In this embodiment, this would be the first of two threads 48 on the inside of the cap 30, located at the inside of the upper part of the cap 30. The test tube 20 has a thread style fastener 46, which allows the test tube 20 to be screwed into the inside threads 50 of the upper section of the cap 30.

In another embodiment, the test tube 20 has the threaded bushing, while the cap 30 has the inserting threads, which allow the cap 30 to be screwed to the test tube.

In some embodiments, the reagent holder 40 can be protruding out from the test tube 20 with the cap 30 covering a portion of this.

In another embodiment the fastener 44 may be a grommet. In another embodiment, the cap 20 may be held to the reagent holder 40 by a friction fit.

In some embodiments, the reagent holder 40 may still be fastened to the cap 30 when the cap 30 is unfastened from the test tube 20. In some embodiments, the cap 30 and reagent holder 40 are fastened together by either the reagent holder 40 having a threaded bushing and the cap 30 having inserting threads or the reagent holder 40 having threads and the cap 30 having an inserting bushing.

In some embodiments, the reagent holder 40 will have a controlled dropper 43. Said controlled dropper 43 will be utilized for the purposes of excreting liquid from the reagent holder 40. The controlled dropper 43 can be either a dropper, a syringe, or another device utilized for excreting a liquid in the reagent holder 40.

In an embodiment, the test tube 20 is transparent to allow the user can see the result of applying the reagent to the specimen. In another embodiment, the test tube 20 is at least transparent enough to allow a user to see the result of applying the reagent to the specimen. In another embodiment, the test tube 20 may have one or more partially transparent windows to allow the user can see the result of applying the reagent to the specimen.

In some embodiments, both the test tube 20 and the reagent holder 40 are transparent enough so that the presence of the ampule 41 can be seen even when the device 10 is in the compact position. This is useful to quickly determine whether the device 10 has been previously used.

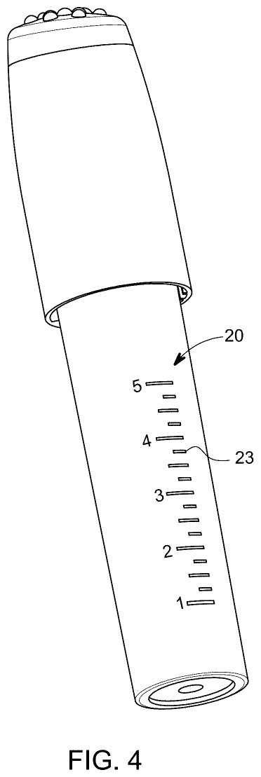

FIG. 4 is a rear perspective view of the device. In this embodiment, the test tube 20 has a quantity measurer 23. The quantity measurer 23 allows the user to determine how much of the specimen and/or the reagent are present. The quantity measurer 23 may be marked in actual volumetric units, such as one milliliter, two milliliters, three milliliters, one cubic centimeter, two cubic centimeters, etc., or may just represent arbitrary volume amounts, i.e., fill to the second line.

The quantity measurer 23 may also be used to measure the desired amount of water to be added to the test tube 20 for a test, for example, adding the correct amount of water for fentanyl testing with an accompanying test strip (not shown).

The cap 30 may also be a predetermined size and used to measure the desired amount of water to be added to the test tube 20 for a test.

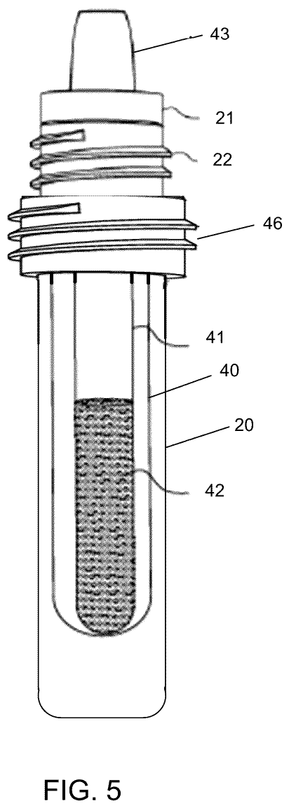

FIG. 5 is a side view of the device 10 without the cap 30. In this embodiment, the test tube 20 is clear with the reagent holder 40 being able to be fully seen. The reagent holder 40 contains at least one ampule 41. The ampule 41 may have an onion skin texture that is able to be broken easily by a user. Inside the ampule 41 is the reagent liquid 42.

In some embodiments, reagent holder 40 is removed from the cap 30 and the test tube 20, the cap 30 can be used to prepare a specimen. If the specimen is in powder form, then the cap 30 can use the measuring end 32 to extract the specimen. Upon extraction of the specimen, the specimen is placed into the test tube 20. Then the user will apply light pressure to the outside of the reagent holder 40 where an ampule 41 is located inside of the reagent holder 40. Upon application of light pressure, the ampule 41 will break, allowing for the reagent liquid 42 to be released from the ampule 41 and into the reagent holder 42. Then, the reagent holder 40 can drop a few drops of reagent liquid 42 from the controlled dropper 43 at an end of the reagent holder 40 into the test tube 20. Upon the contact of the reagent liquid 42 and the specimen, the reagent liquid 42 will change color, based on the specific reagent liquid 42 type, and on the components of the specimen.

In some embodiments, the reagent holder 40 has enough reagent liquid 42 for multiple tests. In an embodiment, the reagent holder 40 has enough reagent liquid 42 for 5 to 10 tests. The multiple tests can be obtained by the reagent holder 40 using the controlled dropper 43, which can place individual drops into the test tube 20. In some embodiments, the multiple tests can be obtained by the user by applying pressure to a portion of the reagent holder 40, which will break the ampule 41, which contains enough reagent liquid 42 for multiple tests.

In other embodiments, there are multiple ampules 41 in the reagent holder 42 to achieve more tests.

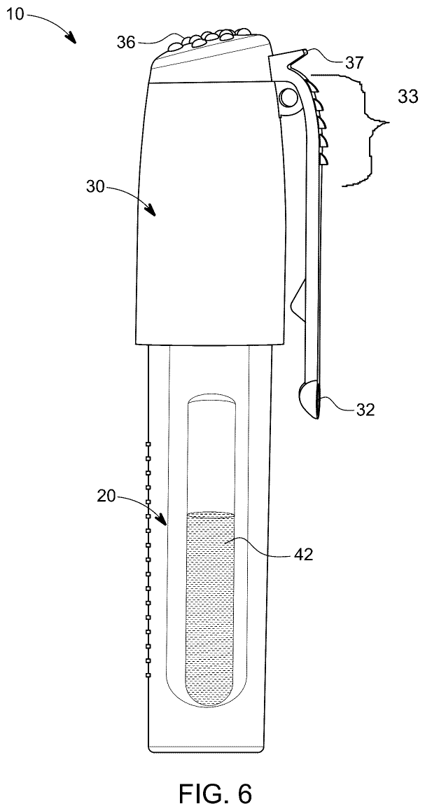

FIG. 6 is a side perspective view of the device with the cap 30 fastened to the test tube 20 according to one or more embodiments of the presently disclosed subject matter. In some embodiments, when the specimen is in pill form, the serrated edge 33 is used to scrape a portion of the outside of the specimen to create a powder. In some embodiments, the serrated edge 33 stores a portion of the scraped portion of the specimen, which can be removed into the test tube 20 for the reagent liquid 42 to mix with the specimen. In some embodiments, when the specimen is in chunk form, the knobby end 35 is able to crush the chunk. The cap 30 can then extract a portion of the specimen that has been crushed by the knobby end 35 by the measuring end 32 or the serrated edge 33 depending on the size or form of the crushed specimen. The extracted portion of the specimen is then placed into the test tube 20 for the reagent liquid 42 to interact with the specimen.

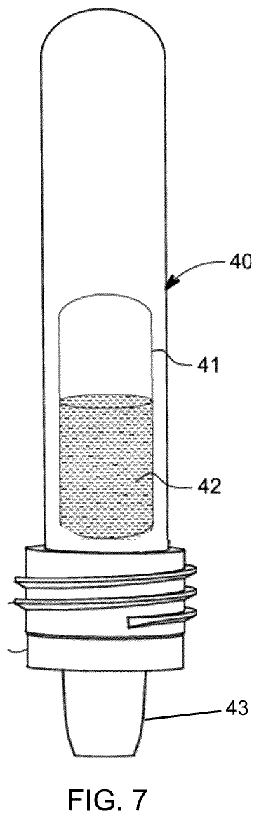

FIG. 7 is a perspective view of one or more embodiments showing an ampule 41 within the reagent holder 40. In some embodiments, there are silica beads or another packing material to store and secure the ampules 41 in the reagent holder 40 and protect the ampules 41 from breaking and leaking the reagent liquid 42 prior to intended use.

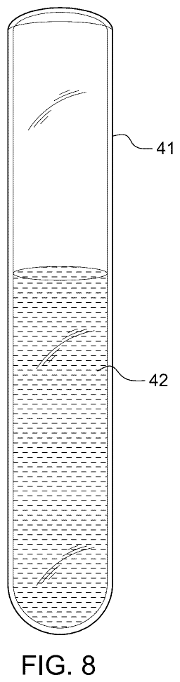

FIG. 8 is a perspective view of one or more embodiments of the ampule 41 with the reagent liquid 42 inside of the ampule 41. The reagent liquid 42 is dependent on what the device 10 is designed to test. A specimen might be a drug or another substance that the user wishes to test, with the color of the testing reaction varying based on what medication or pharmaceutical or other substance is being tested. The chosen reagent liquid 42 in the reagent holder 40 will vary, based on product offerings and the specific medication and pharmaceutical substance it is intended to test.

FIG. 9 is an exploded view of the test tube 20, the cap 30, and the reagent holder 40. In an embodiment, the cap 30 has an internal smaller-diameter threaded section 48, and an internal larger diameter threaded section 50. The reagent holder 40 has a fastener 44, preferably but not necessarily threads, which screw into the smaller-diameter threaded section 48. The test tube 20 has a fastener 46, preferably but not necessarily threads, which screw into the larger-diameter threaded section 50.

In an embodiment, the fastener 44 on the reagent holder 40 is a threaded bushing. In an implementation of this embodiment, the first of two threads of fastener 44 screw into the smaller-diameter threaded section 48. Similarly, the first of two threads of fastener 46 screw into the larger-diameter threaded section 50. More threads may be used if desired for a more secure arrangement.

For storage or transportation, the reagent holder 40 is screwed into the cap 30, and then the test tube 20 is screwed into the cap 30. For use, the test tube 20 is unscrewed from the cap 30, and then the reagent holder 40 is unscrewed from the cap 30.

In another embodiment, the fasteners 44 and/or 48, and/or fasteners 46 and/or 50, may be grommets, in which case threads are not needed. In another embodiment, the cap 30 may be held to the reagent holder 40, and/or to the reagent holder 20, by a friction fit.

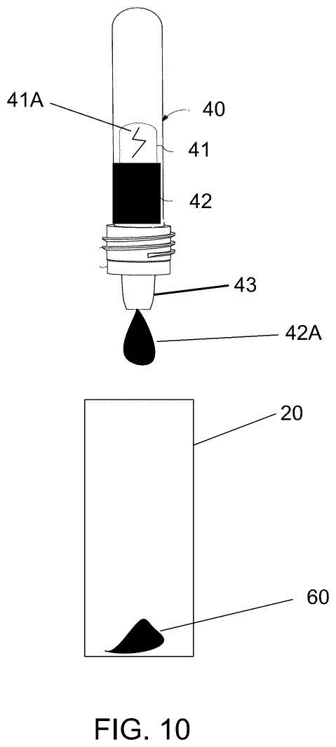

FIG. 10 is an illustration showing one or more drops 42A of the reagent liquid 42 being applied to a substance 60 in the test tube 20. The jagged line 41A merely indicates that the ampule 41 has been broken so that the reagent liquid 42 is available for the test.

In use, when it is desired to test a substance for the presence of an active ingredient, the test tube 20 is removed from the cap 30, the reagent holder 40 may be removed from the cap 30 at this time if desired. If the substance is already in a powdered or finely granulated form, the measuring end 32 is used to measure out a desired quantity of the substance into the test tube 20.

If the substance is not already in a powdered or finely granulated form, such as a pill or tablet, the serrated edge 33 and/or the knobby end 35 is used to grind off or pulverize a quantity of the substance. The measuring end 32 is used to measure out a desired quantity of the substance into the test tube 20.

The substance may also be in a liquid form, in which case the measuring end 32 is used to measure out a desired quantity of the substance into the test tube 20.

In an embodiment, the amount of the substance to be tested is measured based on the lines in the quantity measurer 23.

In another embodiment, the amount of the substance to be tested is measured as one “scoop,” two “scoops,” etc.

In another embodiment, the amount of the reagent liquid 42 to be added to the substance 42A to be tested is measured based on the lines in the quantity measurer 23.

The user then squeezes or bends the reagent holder 40, or takes some other action, to break the ampule 41 inside, thereby releasing the reagent liquid 42 into the reagent holder 40. The user then allows the reagent liquid 42 to drip into the test tube 40 containing the powdered substance and/or squeezes the reagent holder 40 to force the reagent liquid 42 into the test tube 40.

Depending upon the type of the reagent liquid 42 and/or the substance being tested, the user may re-attach the cap 30 to the test tube 20 and shake, so as to assure adequate mixing of the substance and the reagent liquid 42. Also, the user may, if desired, use the clip 31 to at least partially stir the reagent liquid 42/substance mixture.

The user then observes whether the expected color change occurs. In most cases this happens instantly, in other cases a wait of seconds or even minutes may be required.

If the substance contains the active ingredient being tested for then the color change will confirm that the active ingredient is present in an at least a non-insignificant amount.

Preferably, but not necessarily, in an embodiment, a small frame, grid, or mesh, at the base of the controlled dropper 43 blocks fragments of ampule 41 from going into and/or blocking the controlled dropper 43.

In an embodiment, the ampule 41 is surrounded in the reagent holder 40 by a non-reactive material so as prevent or reduce the likelihood of accidental breakage of the ampule 41. A “non-reactive” material is one which will have little or no interaction with the reagent liquid 42 so that it will not affect the color test. An example of a non-reactive material is silica beads.

A non-limiting discussion of various reagent liquids 42 and relevant active ingredients are discussed below.

The amount of the substance to be tested is generally a small amount, such as the size of a pinhead of powder, 1/10 of a blotter, a few crumbs scraped off of a pill, or 1 to 5 mg per test.

In an embodiment, the reagent liquid 42 is 100 ml Sulfuric Acid, 5 ml Formalin, and 5 ml of Methanol. Upon application of this reagent liquid 42 to heroin, the reagent liquid 42 will change color to purple-red.

In another embodiment, the reagent liquid 42 is 100 ml Sulfuric Acid and 1 gram of ammonium metavanadate. Upon application of this reagent liquid 42 to cocaine, the reagent liquid 42 will change color to an orange-yellow color.

In another embodiment, the reagent liquid 42 is 50 ml of Ethanol, 50 ml Hydrochloric Acid, and 2 grams of P-DMAB. Upon application of this reagent liquid 42 to LSD, the reagent liquid 42 will change color to a purplish color.

In another embodiment, the reagent liquid 42 is 65 ml of Sulfuric Acid, 35 ml distilled water, 125 milligrams of P-DMAB, 50 ml of distilled water and 4.5 grams of FeCl₃. Upon application of this reagent liquid 42 to LSD, the reagent liquid 42 will change color to a blue color.

In another embodiment, the reagent liquid 42 is 100 ml of Sulfuric Acid and 1-gram Selenious Acid. Upon application of this reagent liquid 42 to oxycodone, the reagent liquid 42 will change color to an olive color.

In another embodiment, the reagent liquid 42 is 100 ml of distilled water, 4 ml acetaldehyde, and 2-gram of Sodium Nitroprusside. This reagent liquid 42 would be used in tandem with another reagent liquid 42, made of 2 g of sodium carbonate, and 100 ml of distilled water. Upon application of both of these reagent liquids 42 with methamphetamine, the reagent liquid combination will change color to a blue color.

In another embodiment, the reagent liquid 42 is 100 ml of Sulfuric Acid, 10-gram of Sodium Nitrite. Upon application of this reagent liquid 42 to amphetamine, the reagent liquid 42 will change color to an orangish-red color.

In another embodiment, the reagent liquid 42 is 100 ml of Sulfuric Acid and 0.5-gram of Sodium Molybdate. Upon application of this reagent liquid 42 to MDMA, the reagent liquid 42 will change color to a greenish to black color.

In another embodiment, the reagent liquid 42 is 100 ml of deionized water and 2-grams of Cobalt (II) thiocyanate. This reagent liquid would be used in tandem with another reagent liquid, made of 100 ml of deionized water, and 0.4 g NaOH. Upon application of both of these reagent liquids 42 to cocaine, the reagent liquid combination 42 will change color to a bright blue color.

“Changing color” as used herein includes manifesting a color which is different than the original color, and includes the reagent liquid changing color, the substance to be tested changing color, or both.

In some embodiments, a list of instructions can be provided with each device that details what each color change or lack of color change can mean when testing the compounds.

In an embodiment the ampules 41 are stacked inside the reagent holder 40 reagent liquid 42. In another embodiment the ampules 41 may be placed side-by-side inside the reagent holder 40.

The device and method described herein have particular use, but not the only use, in testing for the presence of drugs in life-threatening situations. For example, there may be an emergency call reporting a suspected drug overdose and the medical personnel arrive to find an unconscious or comatose person on the floor with a powder or non-marked pills nearby. Administering the wrong antidote can be deadly, but waiting for a laboratory to test and identify the drug can also be deadly. In accordance with the embodiments described herein, medical personnel can carry a set of these testing devices, each one having a reagent for a particular drug. The medical personnel can then scoop some of the drug into several of the test tubes 20, or use the cap 30 to crush the pills and scoop some of the drug into several of the test tubes 20. The medical personnel can then squeeze various reagent holders 40 to break the ampules 41 within, place some of the released reagent fluid 42 from each reagent holder 40 into a respective test tube 20, and watch for a color change. This can be done quickly, and even performed while the patient is being transported to the hospital. A quick and early diagnosis of the drug involved can therefore save a life.

The device 10 and method described herein therefore provide for a compact, easy to carry, easy to use, versatile test kit for determining the presence of certain substances. The cap 30 and the test tube 20 contain and protect the frangible reagent holder 40. At least a part of the test tube 20 is at least transparent so that a color change can be detected when the reagent 41 is mixed with the substance in the test tube 20. The cap 30 may be used to measure a quantity of a specified liquid to be poured into the test tube 20 for the testing. The cap 30 may have a clip 31 which allows the device 10 to be clipped to a shirt pocket, pants pocket, purse pocket, or other article for easy transport and ready availability. The cap 30 may also have a measuring end 32, which can be used as a scoop, and/or a serrated edge 33 and/or a knobby end 35 for producing a powder or small particles for testing. The reagent holder 40 contains a desired reagent 41 for testing for a particular substance.

The figures and descriptions provided herein may have been simplified to illustrate aspects that are relevant for a clear understanding of the herein described devices, systems, and methods, while eliminating, for the purpose of clarity, other aspects that may be found in typical devices, systems, and methods. Those of ordinary skill may recognize that other elements and/or operations may be desirable and/or necessary to implement the devices, systems, and methods described herein. Because such elements and operations may be well known in the art, and because they do not facilitate a better understanding of the present disclosure, a discussion of such elements and operations is not provided herein. The present disclosure is deemed to inherently include all such elements, variations, and modifications to the described aspects that would be known to those of ordinary skill in the art, particularly in view of reading the present disclosure.

The terminology used herein is for the purpose of describing particular example embodiments only and is not intended to be limiting. As used herein, the singular forms “a”, “an”, and “the” may be intended to include the plural forms as well, unless the context clearly indicates otherwise. The terms “comprises,” “comprising,” “including,” and “having,” are inclusive and therefore specify the presence of stated features, integers, steps, operations, elements, and/or components, but do not preclude the presence or addition of one or more other features, integers, steps, operations, elements, components, and/or groups thereof. The method steps, processes, and operations described herein are not to be construed as necessarily requiring their performance in the particular order discussed or illustrated, unless specifically identified as an order of performance. It is also to be understood that additional or alternative steps may be employed.

When an element or layer is referred to as being “on”, “engaged to”, “connected to” or “coupled to” another element or layer, it may be directly on, engaged, connected or coupled to the other element or layer, or intervening elements or layers may be present. In contrast, when an element is referred to as being “directly on,” “directly engaged to”, “directly connected to” or “directly coupled to” another element or layer, there may be no intervening elements or layers present. Other words used to describe the relationship between elements should be interpreted in a like fashion (e.g., “between” versus “directly between,” “adjacent” versus “directly adjacent,” etc.). As used herein, the term “and/or” includes any and all combinations of one or more of the associated listed items.

Although the terms first, second, third, etc., may be used herein to describe various elements, components, regions, layers and/or sections, these elements, components, regions, layers and/or sections should not be limited by these terms. These terms may be only used to distinguish one element, component, region, layer or section from another element, component, region, layer or section. Terms such as “first,” “second,” and other numerical terms when used herein do not imply a sequence or order unless clearly indicated by the context. Thus, a first element, component, region, layer or section discussed below could be termed a second element, component, region, layer or section without departing from the teachings of the exemplary embodiments.

Unless otherwise defined, all terms (including technical and scientific terms) used herein have the same meaning as commonly understood by one of ordinary skill in the art to which this subject matter belongs. It will be further understood that terms, such as those defined in commonly used dictionaries, should be interpreted as having a meaning that is consistent with their meaning in the context of the specification and relevant art and should not be interpreted in an idealized or overly formal sense unless expressly so defined herein. For brevity and/or clarity, well-known functions or constructions may not be described in detail herein.

The terms “for example” and “such as” mean “by way of example and not of limitation.” The subject matter described herein is provided by way of illustration for the purposes of teaching, suggesting, and describing, and not limiting or restricting. Combinations and alternatives to the illustrated embodiments are contemplated, described herein, and set forth in the claims.

For convenience of discussion herein, when there is more than one of a component, that component may be referred to herein either collectively or singularly by the singular reference numeral unless expressly stated otherwise or the context clearly indicates otherwise. For example, components N (plural) or component N (singular) may be used unless a specific component is intended. Also, the singular forms “a,” “an,” and “the” are intended to include the plural forms as well, un-less expressly stated otherwise or the context indicates otherwise.

The terms “includes,” “has,” “having,” or “exhibits,” or variations in form thereof are intended to be inclusive in a manner similar to the term “comprises” as that term is interpreted when employed as a transitional word in a claim.

It will be understood that when a component is referred to as being “connected” or “coupled” to another component, it can be directly connected or coupled or coupled by one or more intervening components unless expressly stated otherwise or the context clearly indicates otherwise.

The term “and/or” includes any and all combinations of one or more of the associated listed items. As used herein, phrases such as “between X and Y” and “between about X and Y” should be interpreted to include X and Y unless expressly stated otherwise or the context clearly indicates otherwise.

Terms such as “about”, “approximately”, “around”, and “substantially” are relative terms and indicate that, although two values may not be identical, their difference is such that the apparatus or method still provides the indicated or desired result, or that the operation of a device or meth-od is not adversely affected to the point where it cannot perform its intended purpose. As an ex-ample, and not as a limitation, if a height of “approximately X inches” is recited, a lower or high-er height is still “approximately X inches” if the desired function can still be performed or the desired result can still be achieved.

While the terms vertical, horizontal, upper, lower, bottom, top, and the like may be used herein, it is to be understood that these terms are used for ease in referencing the drawing and, unless otherwise indicated or required by context, does not denote a required orientation.

The different advantages and benefits disclosed and/or provided by the embodiment(s) disclosed herein may be used individually or in combination with one, some or possibly even all of the other benefits. Furthermore, not every embodiment, nor every component of an embodiment, is necessarily required to obtain, or necessarily required to provide, one or more of the advantages and benefits of the embodiment.

Conditional language, such as, among others, “can”, “could”, “might”, or “may”, unless specifically stated otherwise, or otherwise understood within the context as used, is generally intended to convey that certain embodiments preferably or optionally include certain features, elements and/or steps, while some other embodiments optionally do not include those certain features, elements and/or steps. Thus, such conditional language indicates, in general, that those features, elements and/or step may not be required for every embodiment.

The subject matter described herein is provided by way of illustration only and should not be construed as limiting the nature and scope of the claims herein. While different embodiments have been provided above, it is not possible to describe every conceivable combination of com-ponents or methodologies for implementing the disclosed subject matter, and one of ordinary skill in the art may recognize that further combinations and permutations that are possible. Furthermore, the nature and scope of the claims is not necessarily limited to embodiments that solve any or all disadvantages which may have been noted in any part of this disclosure. Various modifications and changes may be made to the subject matter described herein without following, or departing from the spirit and scope of, the exemplary embodiments and applications illustrated and described herein. Although the subject matter presented herein has been described in language specific to components used therein, it is to be understood that the scope of the claims is not necessarily limited to the specific components or characteristics thereof described herein; rather, the specific components and characteristics thereof are disclosed as example forms of implementing the disclosed subject matter. Accordingly, the disclosed subject matter is intended to embrace all alterations, modifications, and variations, that fall within the scope and spirit of any claims that may be written therefor.

1. A device to test for the presence of a predetermined substance, comprising:
a cap;
a flexible reagent holder reversibly attached to the cap;
a breakable ampule contained within the reagent holder, the breakable ampule containing a reagent liquid; and
a test tube reversibly attached to the cap and removably containing the flexible reagent holder, the test tube having at least one transparent area wherein a color change can be seen;
wherein the breakable ampule releases the reagent liquid when the flexible reagent holder is manipulated; and
wherein a predetermined color is manifested when the reagent liquid is mixed in the test tube with a powder containing the predetermined substance.

2. The device of claim 1, and further comprising a clip attached to the cap, the clip containing a serrated edge.

3. The device of claim 1, and further comprising a clip attached to the cap, the clip containing a measuring end which measures a predetermined amount.

4. The device of claim 1, wherein the test tube has a set of measuring lines thereon.

5. The device of claim 1, wherein the cap comprises a knobby end, wherein a pill or a tablet of the predetermined substance may be crushed to provide a powder for testing.

6. The device of claim 1, wherein the reagent holder has a first end, the first end being reversibly inserted into the cap, the first end comprising a dropper wherein the reagent liquid may be expelled when the breakable ampule is broken.

7. The device of claim 1, wherein the cap has a set of internal threads and the flexible reagent holder has a set of external threads, wherein the internal threads and the external threads reversibly attach the cap to the flexible reagent holder.

8. The device of claim 1, wherein the cap has a set of internal threads and the test tube has a set of external threads, wherein the internal threads and the external threads reversibly attach the cap to the test tube.

9. The device of claim 1, wherein:
the cap has a first set of internal threads and the flexible reagent holder has a set of external threads which mate with the first set of internal threads; and
the cap has a second set of internal threads and the test tube has a set of external threads which mate with the second set of internal threads.

10. The device of claim 1, wherein the reagent liquid is one of:
100 ml Sulfuric Acid, 5 ml Formalin, and 5 ml of Methanol, and the predetermined color is a purple-red mixture upon application to heroin;
100 ml Sulfuric Acid and 1 gram of ammonium metavanadate, and the predetermined color is an orange-yellow color upon application to cocaine;
50 ml of Ethanol, 50 ml Hydrochloric Acid, and 2 grams of P-DMAB, and the predetermined color is a purplish color upon application to LSD;
65 ml of Sulfuric Acid, 35 ml distilled water, 125 milligrams of P-DMAB, 50 ml of distilled water and 4.5 grams of FeCl₃, and the predetermined color is a blue color upon application to LSD;
100 ml of Sulfuric Acid and 1-gram Selenious Acid, and the predetermined color is an olive color upon application to oxycodone;
100 ml of distilled water, 4 ml acetaldehyde, and 2-gram of Sodium Nitroprusside, combined with 2 g of sodium carbonate, and 100 ml of distilled water, and the predetermined color is a blue color upon application to methamphetamine;
100 ml of Sulfuric Acid, 10-gram of Sodium Nitrite, and the predetermined color is an orangish-red color upon application to amphetamine;
100 ml of Sulfuric Acid and 0.5-gram of Sodium Molybdate, and the predetermined color is a greenish to black color upon application to MDMA; or
100 ml of deionized water and 2-gram of Cobalt (II) thiocyanate, combined with 100 ml of deionized water and 0.4 g NaOH, and the predetermined color is a bright blue color upon application to cocaine.

11. A method to test for the presence of a predetermined substance, comprising:
obtaining a powder of a substance to be tested;
using a measuring device on a cap of a testing device to provide a measured quantity of the powder;
placing the measured quantity into a test tube of the testing device;
manipulating a reagent holder of the testing device to break an ampule contained within the reagent holder to release a reagent liquid;
squeezing the reagent holder to expel at least some of the reagent liquid onto the powder in the test tube; and
looking at or into the test tube to determine that a predetermined color is manifested when the reagent liquid is mixed with the powder.

12. The method of claim 11, wherein looking at or into the test tube comprises looking into a transparent area on the test tube.

13. The method of claim 11, wherein obtaining the powder of the substance to be tested comprises using a serrated edge on the cap of the testing device to scrape a pill or tablet.

14. The method of claim 11, wherein obtaining the powder of the substance to be tested comprises using a set of small knobs on the top of the cap of the testing device to crush a pill or tablet.

15. The method of claim 11, wherein manipulating the reagent holder comprises squeezing the reagent holder to break the ampule.

16. The method of claim 11, and further comprising declaring the predetermined substance to be present if the predetermined color is manifested.

17. The method of claim 11, wherein the reagent liquid is one of:
100 ml Sulfuric Acid, 5 ml Formalin, and 5 ml of Methanol, and the predetermined color is a purple-red mixture upon application to heroin;
100 ml Sulfuric Acid and 1 gram of ammonium metavanadate, and the predetermined color is an orange-yellow color upon application to cocaine;
50 ml of Ethanol, 50 ml Hydrochloric Acid, and 2 grams of P-DMAB, and the predetermined color is a purplish color upon application to LSD;
65 ml of Sulfuric Acid, 35 ml distilled water, 125 milligrams of P-DMAB, 50 ml of distilled water and 4.5 grams of FeCl₃, and the predetermined color is a blue color upon application to LSD;
100 ml of Sulfuric Acid and 1-gram Selenious Acid, and the predetermined color is an olive color upon application to oxycodone;
100 ml of distilled water, 4 ml acetaldehyde, and 2-gram of Sodium Nitroprusside, combined with 2 g of sodium carbonate, and 100 ml of distilled water, and the predetermined color is a blue color upon application to methamphetamine;
100 ml of Sulfuric Acid, 10-gram of Sodium Nitrite, and the predetermined color is an orangish-red color upon application to amphetamine;
100 ml of Sulfuric Acid and 0.5-gram of Sodium Molybdate, and the predetermined color is a greenish to black color upon application MDMA; or
100 ml of deionized water and 2-gram of Cobalt (II) thiocyanate, combined with 100 ml of deionized water and 0.4 g NaOH, and the predetermined color is a bright blue color upon application to cocaine.

18. A device to test for the presence of a predetermined substance, comprising:
a cap having a knobby end;
a clip attached to the cap, the clip comprising a serrated edge;
wherein the knobby end is used to crush a pill or a tablet, or the serrated edge is used to scrape a pill or a tablet, to provide a powder;
a flexible reagent holder reversibly attached to the cap, the flexible reagent holder having a first end, the first end being reversibly inserted into the cap, the first end comprising a dropper wherein the reagent liquid may be expelled when a breakable ampule is broken;
the breakable ampule being contained within the reagent holder and containing a reagent liquid; and
a test tube having a set of measuring lines thereon, reversibly attached to the cap, and removably containing the flexible reagent holder, the test tube having at least one transparent area wherein a color change can be seen;
the clip further comprising a measuring end to measure a measured amount of the powder into the test tube;
wherein the breakable ampule releases the reagent liquid onto the powder in the test tube when the flexible reagent holder is manipulated; and
wherein a predetermined color is manifested when the reagent liquid is mixed in the test tube with the powder if the powder contains the predetermined substance.

19. The device of claim 18, wherein:
the cap has a first set of internal threads and the flexible reagent holder has a set of external threads which mate with the first set of internal threads; and
the cap has a second set of internal threads and the test tube has a set of external threads which mate with the second set of internal threads.

20. The device of claim 18, wherein the reagent liquid is one of:
100 ml Sulfuric Acid, 5 ml Formalin, and 5 ml of Methanol, and the predetermined color is a purple-red mixture upon application to heroin;
100 ml Sulfuric Acid and 1 gram of ammonium metavanadate, and the predetermined color is an orange-yellow color upon application to cocaine;
50 ml of Ethanol, 50 ml Hydrochloric Acid, and 2 grams of P-DMAB, and the predetermined color is a purplish color upon application to LSD;
65 ml of Sulfuric Acid, 35 ml distilled water, 125 milligrams of P-DMAB, 50 ml of distilled water and 4.5 grams of FeCl₃, and the predetermined color is a blue color upon application to LSD;
100 ml of Sulfuric Acid and 1-gram Selenious Acid, and the predetermined color is an olive color upon application to oxycodone;
100 ml of distilled water, 4 ml acetaldehyde, and 2-gram of Sodium Nitroprusside, combined with 2 g of sodium carbonate, and 100 ml of distilled water, and the predetermined color is a blue color upon application to methamphetamine;
100 ml of Sulfuric Acid, 10-gram of Sodium Nitrite, and the predetermined color is an orangish-red color upon application to amphetamine;
100 ml of Sulfuric Acid and 0.5-gram of Sodium Molybdate, and the predetermined color is a greenish to black color upon application MDMA; or
100 ml of deionized water and 2-gram of Cobalt (II) thiocyanate, combined with 100 ml of deionized water and 0.4 g NaOH, and the predetermined color is a bright blue color upon application to cocaine.
